(19) United States
(12) Patent Application Publication
(10) Pub. No.: US 2015/0079042 A1
(43) Pub. Date: Mar. 19, 2015

(54) Natural Pet Treat and Method of Production

(71) Applicants: Doris Foley, Cincinnati, OH (US); Daniel Harper, Cincinnati, OH (US)

(72) Inventors: Doris Foley, Cincinnati, OH (US); Daniel Harper, Cincinnati, OH (US)

(21) Appl. No.: 14/469,043

(22) Filed: Aug. 26, 2014

Related U.S. Application Data
(60) Provisional application No. 61/878,897, filed on Sep. 17, 2013.

(51) Int. Cl. A61K 36/54; A23K 1/16; A23K 1/00; A61K 35/74; A61K 47/46; A23K 1/18; A61K 36/45

(52) U.S. Cl. CPC A61K 36/54; A23K 1/1846; A23K 1/1646; A61K 36/45; A61K 35/741; A61K 47/46; A23K 1/001
USPC 424/93.4; 264/163

(57) ABSTRACT

The invention disclosed herein is directed to a pet treat composition and the method of producing such a composition. The pet treat comprises a mixture of whole wheat flour, blueberries, cinnamon, vanilla yogurt, and almond milk. The composition includes organic and natural ingredients and contains a high level of antioxidants, which provides anti-inflammatory properties that help prevent joint and hip pain. Additionally, the composition contains probiotics that enhance digestive function. When consumed, the probiotics have shown to help the chemical balance and reduce tearing as well as the bacteria that causes tear stains. The present method generally involves mixing all of the ingredients together to form a dough and baking the dough. The composition is suitable for daily consumption and as supplement to a pet's existing diet regimen.

### CROSS REFERENCE TO RELATED APPLICATION

This application claims the benefit of U.S. Provisional Application No. 61/878,897 filed on Sep. 17, 2013. The above identified patent application is herein incorporated by reference in its entirety to provide continuity of disclosure.

### BACKGROUND OF THE INVENTION

1. Field of the Invention

The present invention relates to a pet treat composition. More specifically, the present invention pertains to pet treats containing wheat flour, blueberries, cinnamon, and yogurt, as well as methods of producing such pet treats. The present pet treats are rich in antioxidants and provide anti-inflammatory properties that help to prevent joint and hip pain for pets.

Good nutrition enhances pets' quality and quantity of life, and is an essential aspect of animal care. Incorporating nutritional assessment into regular animal care is critical to maintaining pets' health, as well as their responses to diseases and injury. Conventional pet treats, however, contain preservatives, chemical additives and fillers. When fed daily for an extended period of time, artificial or synthetic food preservatives could be toxic to pets.

There has been an increasing interest in holistic and natural diets for pets. Although many commercial pet food companies pledge to use all-natural, human grade ingredients, some food products are not made in USDA and FDA approved facilities in the United States. Additionally, pet food products are not as highly regulated as food products for human consumption. Accordingly, commercial pet foods have an increased likelihood of contamination and spread of diseases. Lastly, many commercial pet food products are costly to the pet owners.

The present invention provides a dog treat that is made of organic, natural ingredients. The present dog treat contains wheat flour, blueberries, cinnamon, vanilla yogurt, and almond milk. The method of producing the present dog treat generally comprises the steps of combining all of the ingredients to form a dough and baking the dough in desired shapes. The present dog treat provides nutritional snacks for dogs while providing a good source of antioxidants that can help reduce inflammation and joint pains associated therewith.

2. Description of the Prior Art

Food compositions containing organic wheat flour, blueberries, cinnamon, and yogurt are currently available commercially in the United States and have been disclosed in the prior art. These include food compositions and methods of production that have been patented and published in patent application publications, and generally relate to food products for human consumption. The following is a list of prior art compositions and methods deemed most relevant to the present disclosure, which are herein described for the purposes of highlighting and differentiating the unique aspects of the present invention, and further highlighting the drawbacks existing in the prior art.

One such device, U.S. Pat. No. 6,419,911 to Bailey discloses a psyllium containing snack bar and the processes for making such snack bars. The snack bars are used in a therapeutic or dietary regimen, for purposes such as increasing dietary fiber or for reducing cholesterol. While Bailey discloses a snack bar that provides therapeutic benefits associated therewith, the purpose and intent of Bailey is to provide a snack bar that incorporates psyllium to yield an organoleptically acceptable product and that is suitable for human consumption. In contrast, the present invention provides a composition for a pet treat, and in particular, a treat for dogs. Additionally, the present invention contains antioxidants that reduces inflammation in joints. In this way, the present invention helps prevent joint and hip pains for pets.

U.S. Published Patent Application Number 2003/0207004 to Cool discloses baby food compositions containing egg yolk and methods of producing the same. The baby food composition comprises approximately 5 to 25 percent egg yolk solids and an acidulant, wherein the acidulant may comprise lactic acid or a fruit or vegetable component. Cool, however, does not disclose a pet treat composition with a high content of antioxidants. Rather, Cool discloses a baby food composition with egg yolk solids.

U.S. Published Patent Application Number 2013/0189392 to Gorris discloses a food product that is designed to deliver a large dose of cinnamon or other additives such as fruit extract or rind to provide medicinal benefit from the cinnamon or other additives. Similar to the foregoing prior art compositions, the food product of Gorris is designed for human consumption. Thus, the dosage of cinnamon that provides medicinal benefit for humans is not necessarily appropriate for pets.

These compositions and methods of producing pet treats disclosed in the prior art have several known drawbacks. These compositions and methods relate to food products for human consumption. The present invention overcomes these limitations by disclosing a pet treat that is high in antioxidants. The pet treats of the present invention may be used to prevent hip and joint pains, as well as to increase the overall health of the pet. It is therefore submitted that the present invention is substantially divergent in elements from the prior art, and consequently it is clear that there is a need in the art for an improvement to existing pet treats and method of production. In this regard, the instant invention substantially fulfills these needs.

### SUMMARY OF THE INVENTION

In view of the foregoing disadvantages inherent in the known types of pet treats and methods of production now present in the prior art, the present invention provides a new and improved pet treat composition and method of production wherein the same can be utilized for providing nutritional benefits and preventing joint pain for pets.

It is therefore an object of the invention to provide a new and improved pet treat composition and method of producing the same that has all of the advantages of the prior art and none of the disadvantages.

Another object of the present invention is to provide a new and improved pet treat composition that comprises natural and organic ingredients.

Yet another object of the present invention is to provide a new and improved pet treat composition that is rich in antioxidants in order to reduce inflammation in joints and eliminate or reduce pain associated therewith.

Still yet another object of the present invention is to provide a new and improved pet treat composition that helps to provide a shiny and soft coat.

Still yet another object of the present invention is to provide a new and improved pet treat composition that helps to reduce or prevent tear stains.

Still yet another object of the present invention is to provide a new and improved pet treat composition that helps older pets improve memory and cognitive function.

Other objects, features, and advantages of the present invention will become apparent from the following detailed description.

### DETAILED DESCRIPTION OF THE INVENTION

For the purposes of presenting a brief and clear description of the present invention, the preferred embodiment will be discussed as used for providing nutritional benefits and preventing joint pain for pets. The formulation ranges for the finished product which constitute the present invention are presented in the following example.

The pet treat composition of the present invention is a solid compound comprising whole wheat flour, blueberries, cinnamon, vanilla yogurt, and almond milk. In a preferred embodiment, all natural or organic ingredients are used to avoid conventional pesticides, fertilizers containing synthetic ingredients or sewage sludge, bioengineering or ionizing radiation.

Blueberries are packed with antioxidants to help maintain a strong immune system and a healthy heart. Blueberries are also known to boost memory and cognitive skills for both humans and animals alike. Blueberries contain fiber, vitamin K, and manganese. Some studies have found that blueberries help to reduce stroke damage in pets and heart disease. Blueberries are rich in natural antioxidants, which play an important role in preventing an increase of oxidative damage in brain aging.

Cinnamon is beneficial for dogs suffering from painful joints due to arthritis. Cinnamon has anti-inflammatory properties that help to reduce joint stiffness and also help ease the pain associated with musculoskeletal disorders. Cinnamon is also good for the heart as its calcium and fiber content is effective in fighting heart disease. Cinnamon is noted to improve blood circulation because of its blood thinning compound content. With good blood circulation, the supply of oxygen to the different cells of the body is ensured giving the dog the capability for higher metabolic activity. Cinnamon has antifungal and antibacterial properties thus it is highly effective in dealing with external and internal infections.

Yogurt is a good source of calcium and zinc for pets. Yogurt contains high amounts of probiotics that help prevent digestive issues and gastrointestinal problems such as diarrhea, infections, inflamed bowel, bacterial overgrowth, or other absorption issues. Probiotics have also shown to help the chemical balance and reduce the tearing as well as the bacteria that causes tear stains. In this way, yogurt may help prevent infection and boost immunity.

In one embodiment of the present invention, dog treats are prepared using the following ingredients:

| | |
|---|---|
| Frozen Blueberries | 340 g |
| Ground Cinnamon | 2.3 g |
| Vanilla Yogurt | 490 g |
| Almond Milk | 480 g |
| All-Natural Whole Wheat Flour | 960 g |

Commercial vanilla yogurt may be used or the vanilla yogurt may be prepared ahead of time. To prepare the vanilla yogurt, approximately 946 ml milk is heated to 185 degrees Fahrenheit, then cooled to approximately 110 degrees Fahrenheit via a cold water bath. Meanwhile, a starter yogurt is warmed to a room temperature, then added to the cooled milk. Alternatively, freeze-dried bacteria may be added to the cooled milk and stirred. The milk mixture is incubated at approximately 100 degrees Fahrenheit to encourage bacterial growth. The milk mixture may be incubated for approximately two to twelve hours, depending on the firmness of the yogurt consistency. Optionally, the resulting yogurt may be strained through cheesecloth for a thicker consistency. Thereafter, organic vanilla extract may be added to the yogurt to develop a vanilla flavor.

Similarly, commercial almond milk may be used or the almond milk may be prepared ahead of time. To prepare the almond milk, approximately one cup of whole almonds is placed in a bowl and covered with filtered or distilled water. The almonds are soaked for 8 to 48 hours, then drained from the soaking water and rinsed with fresh, cool running water. The almonds are blended with two cups of filtered or distilled water at a high speed for approximately two to four minutes, or until the almonds are broken down into a very fine meal and the water is white and opaque. The blended almond and water mixture is then strained through a cheese cloth or a mesh strainer. The strained solution yields approximately 480 g of almond milk.

A method for producing the pet treat composition of the present invention includes the steps of blending frozen blueberries with vanilla yogurt, cinnamon, and almond milk until the mixture reaches a consistent texture. The mixture is poured into a bowl and added to the wheat flour. All of the ingredients are combined at room temperature until the composition yielded a light, sticky dough. The dough is then placed on a flat surface and rolled out until it is approximately 0.5 inches in thickness. The dough can then be cut into desired shapes and baked for approximately 30 minutes at 350 degrees Fahrenheit in an oven. When the treats are prepared, the treats can be immediately consumed or refrigerated or frozen for later consumption.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above descriptions then, it is to be realized that the optimum dimensional relationships for the parts of the invention, to include variations in size, materials, shape, form, function, and manner of operation, assembly and use, are deemed readily apparent and obvious to one skilled in the art, and all equivalent relationships to those illustrated in the drawings and described in the specifications are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A pet treat composition adapted to reduce inflammation, consisting essentially of:
whole wheat flour in an amount of approximately 42.2% by weight;
frozen blueberries in an amount of approximately 15.0% by weight;
cinnamon in an amount of approximately 0.1% by weight;
yogurt in an amount of approximately 21.6% by weight;
almond milk in an amount of approximately 21.1% by weight;
wherein said whole wheat flour, said frozen blueberries, said cinnamon, said yogurt, and said almond milk are mixed together to form a dough;
wherein said dough is baked.

2) The pet treat composition of claim 1, wherein said yogurt is vanilla flavored.

3) A method of preparing a pet treat, comprising the steps of:
blending frozen blueberries, cinnamon, yogurt; and almond milk at a room temperature to create a smooth mixture;
combining said smooth mixture to whole wheat flour;
mixing said smooth mixture and said whole wheat flour to create a dough;
rolling said dough into a thin sheet;
cutting out said dough into desired shapes;
baking said dough.

4) The method of preparing a pet treat of claim 3, wherein said room temperature is 62 degrees to 73 degrees Fahrenheit.

5) The method of preparing a pet treat of claim 3, wherein:
a final weight percentage of said whole wheat flour is: 42.2%;
a final weight percentage of said frozen blueberries is 15.0%;
a final weight percentage of said cinnamon 0.1% a final weight percentage of said yogurt is 21.6%;
a final weight percentage of said almond milk is 21.1%.

6) The method of preparing a pet treat of claim 3, wherein said yogurt is vanilla yogurt.
